(19) United States
(12) Patent Application Publication
(10) Pub. No.: US 2008/0241446 A1
(43) Pub. Date: Oct. 2, 2008

(54) Composite material and methods of filament winding, pultrusion and open molding that material

(76) Inventors: Leonard J. Adzima, Pickerington, OH (US); Richard P. Krumlauf, Thornville, OH (US); Michael Strait, Johnstown, OH (US); Teresa Wagner, Granville, OH (US); Mark Greenwood, Granville, OH (US); Kevin Spoo, Newark, OH (US)

Correspondence Address: OWENS CORNING, 2790 COLUMBUS ROAD, GRANVILLE, OH, 43023, US

(21) Appl. No.: 11/729,177

(22) Filed: Mar. 28, 2007

(51) Int. Cl. B32B 1/08; A61F 6/06; B29D 22/00

(52) U.S. Cl. 428/34.5; 264/172.11; 264/642; 424/430

(57) ABSTRACT

A composite material is provided. The composite material includes a continuous roving made from a material selected from a group consisting of natural fibers, mineral fibers, synthetic fibers, kenaf fibers, hemp fibers, carbon fibers, glass fibers, aramid fibers and mixtures thereof impregnated with a water-based matrix binder including gypsum, a polymer and water.

### TECHNICAL FIELD AND INDUSTRIAL APPLICABILITY OF THE INVENTION

The present invention relates generally to the composite material field and, more particularly, to a novel composite material, a method of manufacturing that material, methods of filament winding, pultrusion and open molding that material and novel products made by that material and those methods.

### BACKGROUND OF THE INVENTION

U.S. Pat. No. 6,811,877 to Haislet et al discloses a cord structure for use in, for example, making tires. The cord structure includes a series of core filaments formed from metallic material, preferably steel, and a series of fiber filaments formed from glass, carbon, polypropylene, nylon, aramid, cotton, wool, lycra or other metallic wires. The filament may be impregnated with various resins including polymer modified cements or gypsum.

U.S. Pat. No. 6,524,679 to Hauber et al discloses a glass reinforced gypsum board comprising randomly oriented glass fiber impregnated with a gypsum slurry.

While it is known to utilize fiber glass impregnated with a polymer modified gypsum matrix to produce a reinforcement for tires and gypsum board for building, the present invention relates to a novel composite material useful in filament winding, pultrusion and open molding processes to produce new and useful products.

### SUMMARY OF THE INVENTION

In accordance with the purposes of the present invention as described herein, an improved composite material is provided comprising a continuous roving impregnated with a water-based matrix binder. The continuous roving is made from a material selected from a group consisting of natural fibers, mineral fibers, synthetic fibers, kenaf fibers, hemp fibers, carbon fibers, glass fibers, aramid fibers and mixtures thereof. While substantially any type of glass fibers may be utilized, E-glass fibers are particularly useful in the composite material.

The water-based matrix binder includes an organic material, an inorganic material and water. Typically the organic material is a polymer selected from a group consisting of urea formaldehyde resin, melamine formaldehyde resin, phenol-formaldehyde resin, polyvinyl acetate, polyvinyl alcohol, styrene butadiene, acrylic emulsion, urethane and mixtures thereof. Typically the inorganic material is a gypsum. The water-based matrix binder includes between about 30 and about 80 weight percent gypsum, between about 0.1 and about 40 weight percent polymer and about 10 and about 30 weight percent water. Typically the water-based matrix binder includes between about 0.1 and about 5 weight percent density reducer. That density reducer may be perlite. Further, the water-based matrix binder may also include between about 0.1 and about 0.5 weight percent polyacrylamide and between about 0.1 and about 0.5 weight percent silane coupling agent. Typically the composite material comprises between about 10 and about 70 weight percent continuous roving and between about 30 and about 90 weight percent water-based matrix binder.

In accordance with an additional aspect of the present invention a method of manufacturing a composite material is provided. That method comprises impregnating a continuous roving made from a material selected from a group consisting of natural fibers, mineral fibers, synthetic fibers, glass fibers, kenaf fibers, hemp fibers, carbon fibers, aramid fibers and mixtures thereof with a water-based matrix binder including an organic material, an inorganic material and water. More specifically, the method includes using a water-based matrix binder including gypsum, polymer and water. Still further, the method includes using a wet glass fiber roving for the continuous roving and a matrix binder including gypsum, polymer and water for the water-based matrix binder. Still further the method includes using the water-based matrix binder with a density of between about 1.2 and about 2.0 grams per cubic centimeter. In addition, the method includes using polyacrylamide in the water-based matrix binder as a processing agent.

In accordance with additional aspects of the present invention, a method of filament winding a product is provided. That method comprises feeding a continuous roving made from a material selected from a group consisting of natural fibers, mineral fibers, synthetic fibers, glass fibers, kenaf fibers, hemp fibers, carbon fibers, aramid fibers and mixtures thereof impregnated with a water-based matrix material including organic material, inorganic material and water and winding that continuous roving over an object selected from a group consisting of a mandrel, a form, a pipe, a vessel, a tank and an epoxy and polymer body. The method may be further described as including using a wet glass fiber roving with a continuous roving and a matrix binder including gypsum, polymer and water for the water-based matrix binder. Further, the method may include rotating the object during winding.

In accordance with yet another aspect of the present invention a method is provided for open molding a product. That method includes placing a continuous roving impregnated with a water-based matrix binder into an open mold, allowing the continuous roving to at least partially set in the open mold to form the product and removing the product from the open mold.

Still further, the present invention may include a pultrusion method. The pultrusion method comprises applying a water-based matrix binder to a continuous roving to form a composite material, drawing the composite material through a die to form a product of a desired cross-section and curing the product. In addition, the method may include using a wet glass fiber continuous roving. Further, the applying step may be further defined as including passing the continuous roving through a dip tank holding the water-based matrix binder including gypsum, a polymer and water.

In accordance with yet another aspect of the present invention a method of making a product is provided. That method comprises forming the product from an epoxy filament material and overwrapping the product with a continuous roving impregnated with a matrix-binder including gypsum, polymer and water. Still further, the forming and/or overlapping steps may further include a filament winding process.

In addition, the present invention includes a pipe comprising a tubular body made from an epoxy filament material overwrapped with a continuous roving and impregnated with a matrix binder including gypsum, polymer and water. Further, the present invention may include a vessel comprising a hollow body made from an epoxy filament material overwrapped with a continuous roving impregnated with a matrix-binder including gypsum, polymer and water.

In the following description there is shown and described several preferred embodiments of this invention, simply by way of illustration of some of the modes best suited to carry out the invention. As it will be realized the invention is capable of other different embodiments and its several details are capable of modification in various aspects all without departing from the invention. Accordingly, the drawings and descriptions will be regarded as illustrative in nature and not as restrictive.

### BRIEF DESCRIPTION OF THE DRAWINGS

The accompanying drawings incorporated in and forming a part of this specification, illustrate several aspects of the present invention, and together with the description serve to explain the principles of the invention. In the drawings:

FIG. 1 is a perspective view of the composite material of the present invention;

FIG. 2 is a schematic illustration of a filament winding apparatus used in the method of the present inventions;

FIG. 3 is a schematic illustration of a pultrusion apparatus used in the method of the present invention;

FIG. 4 is a perspective view of an open mold used in the method of the present invention;

FIG. 5 is an end elevational view of a pipe constructed in accordance with the present invention; and

FIG. 6 is a partially cut away perspective view illustrating a vessel constructed in accordance with the present invention.

Reference will now be made in detail to the present preferred embodiments of this invention, examples of which are illustrated in the accompanying drawing figures.

### DETAILED DESCRIPTION OF THE PREFERRED EMBODIMENTS OF THE INVENTION

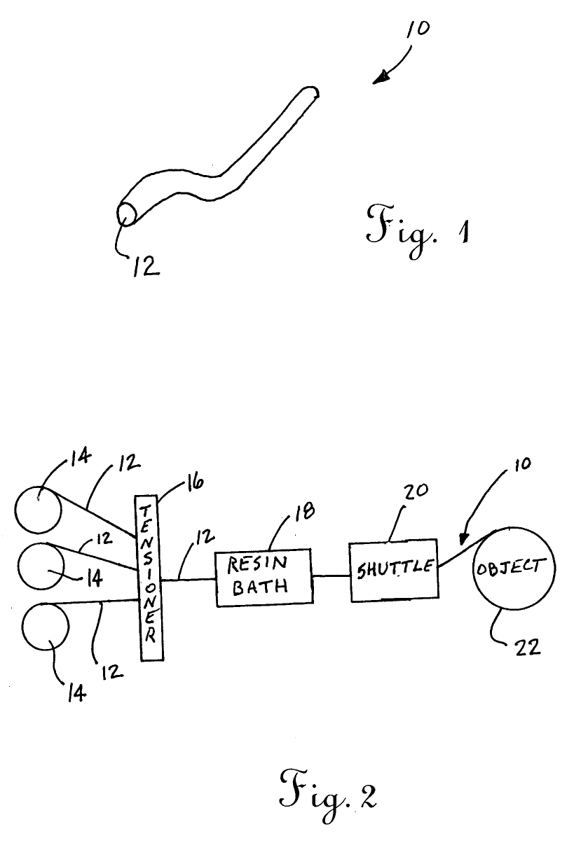

Reference is now made to FIG. 1 illustrating the composite material 10 of the present invention. The composite material 10 comprises a continuous roving 12, such as a T-30 single end roving, impregnated with a water-based matrix binder. More specifically, the continuous roving is made from a material selected from a group consisting of natural fibers, mineral fibers, synthetic fibers and mixtures thereof. More typically the continuous roving is constructed from kenaf fibers, hemp fibers, carbon fibers, glass fibers, aramid fibers and mixtures thereof. Substantially any type of glass fibers may be utilized for the continuous roving but E-glass fibers and S2-glass fibers, including those sold under the trademark Advantex, are particularly useful for this purpose. Typically the fibers in the roving have a diameter of between about 13 and about 23 microns with 16 microns being particularly useful.

The water-based matrix binder may be broadly described as including an organic material, an inorganic material and water. The organic material is typically a polymer. That polymer may be selected from a group consisting of urea formaldehyde resin, melamine formaldehyde resin, phenol-formaldehyde resin, polyvinyl acetate, polyvinyl alcohol, styrene butadiene, acrylic emulsion, urethane and mixtures thereof. The inorganic material is typically gypsum. That gypsum may comprise alpha gypsum, beta gypsum or any mixture thereof. Typically the water-based matrix binder includes between about 30 and about 80 weight percent gypsum, between about 0.1 and about 40 weight percent polymer and between about 10 and about 30 weight percent water. The water-based matrix binder typically has a density of between about 1.2 and about 2.0 grams per cubic centimeter. The matrix binder may include between about 0.1 and about 5 weight percent density reducer. Perlite is particularly useful as a density reducer in the present invention. Further the water-based matrix binder may also include between about 0.1 and about 0.5 weight percent polyacrylamide. The polyacrylamide functions as a processing agent and aids in the wetting of the continuous roving 12 and the impregnation of the matrix binder into the filaments and fibers of that roving. Still further the water-based matrix binder also includes between about 0.1 and about 0.5 weight percent silane coupling agent. Typically the composite material comprises between about 10 and about 70 weight percent continuous roving and between about 30 and about 90 weight percent water-based matrix binder.

The composite material 10 is manufactured by impregnating the continuous roving 12 with the water-based matrix binder. This may be done in a number of ways including, for example, feeding the continuous roving 12 through a dip tank containing the matrix-binder material. Since the matrix binder is water-based, the roving may be fed wet or dry. The ability to feed and properly impregnate a wet continuous roving represents a significant manufacturing advantage as it allows one to eliminate the step of drying the roving before further processing. This significantly reduces both processing times and production costs.

Reference is now made to FIG. 2 schematically illustrating a method of filament winding a product. As illustrated, a series of dry or wet continuous rovings 12 are fed from the creels 14 through a tensioner 16. The rovings 12 are then drawn through a resin bath 18 wherein the resin matrix impregnates the rovings 12 to form the composite material 10. The composite material is then fed through a shuttle 20 before being wound onto an object 22. That object 22 may be, for example, a mandrel, a form, a pipe, a vessel, a tank or an epoxy and polymer body so long as that body does not include reentrant curvature.

The composite material 10 may be wound in a manner well known in the art. Thus, the composite material 10 may be wrapped in adjacent bands or in repeating bands that are stepped the width of the band until it eventually covers the surface of the object 22. Local reinforcement may be added to the structure during circumferential winding, local helical bands, or by the use of woven or unidirectional clothe. The wrap angle may be varied from about 0 to about 90 degrees relative to the axis of the object being wound in a manner known in the art.

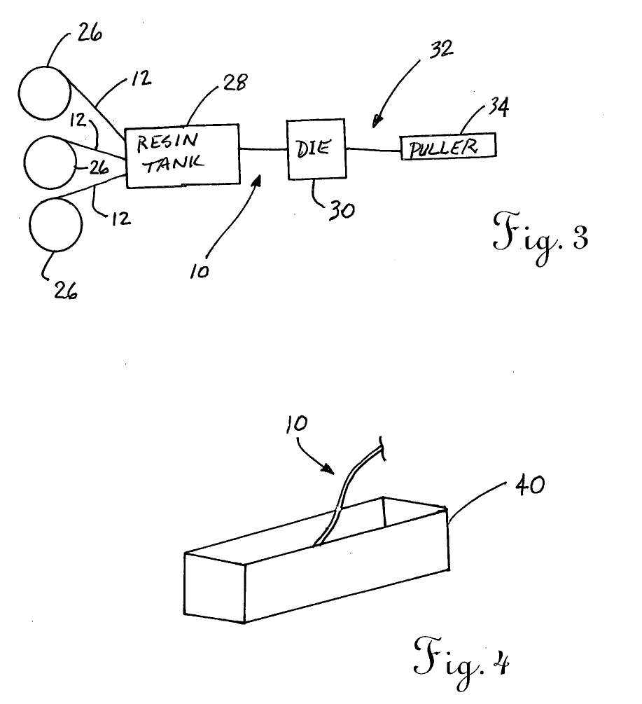

Reference is now made to FIG. 3, schematically illustrating the pultrusion method of the present invention. As illustrated, a series of continuous rovings 12 are fed from creels 26 through a resin tank 28 containing the matrix binder. There the matrix binder impregnates the rovings 12 before the resulting composite material 10 is drawn by a puller 34 through a die 30 which causes the matrix binder to hydrate and harden to produce a product 32 of desired cross-section. This is followed by the curing of that product 32.

FIG. 4 illustrates the open molding process of the present invention. More specifically, the composite material 10 of the present invention, consisting of a continuous roving impregnated with a water-based matrix binder is placed into the open mold 40. The composite material 10 is then allowed to partially set in the open mold to form the product. The setting process may be significantly shortened by heating the open mold. The product is then removed from the open mold.

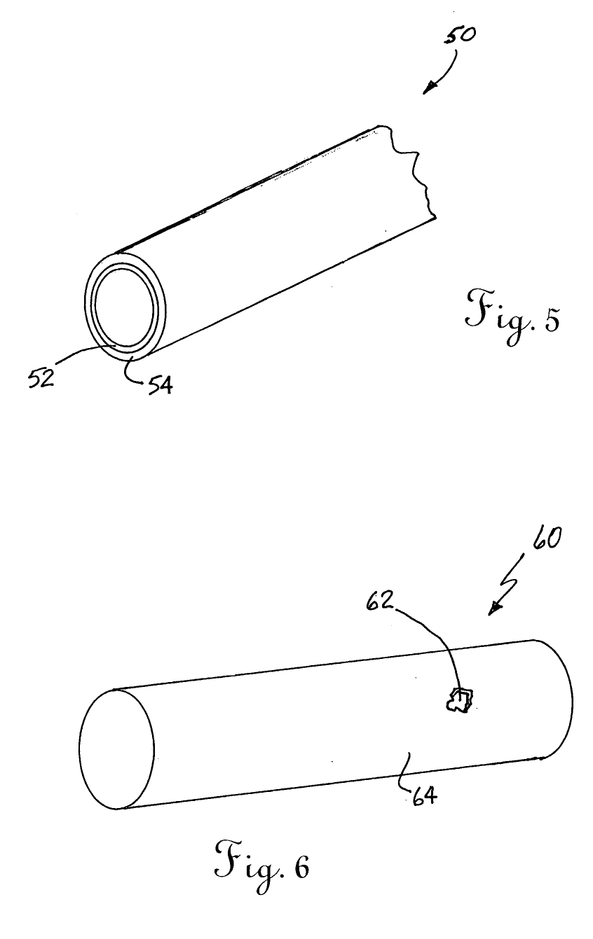

Various products may be made utilizing the composite material 10 of the present invention. FIG. 5 illustrates a pipe 50 that may be constructed using the composite material 10. More specifically, the pipe 50 includes a tubular body 52 made from an epoxy filament material in a manner well known in the art. The tubular body 52 is overwrapped with a layer 54 of the composite material 10 of the present invention comprising continuous roving 12 impregnated with a matrix-binder including gypsum, polymer and water.

FIG. 6 discloses a vessel 60 constructed using the composite material 10 of the present invention. More specifically, the vessel 60 includes a hollow body 62 made, for example, from an epoxy filament material in a manner well known in the art. That body 62 is overwrapped with a layer 64 of composite material of the present invention comprising a continuous roving 12 impregnated with a matrix-binder including gypsum, polymer and water. The vessel 60 may comprise a pressure vessel or even a water tank of the type utilized for a hot water heater.

The foregoing description of the preferred embodiments of the present invention has been presented for purposes of illustration and description. It is not intended to be exhaustive or to limit the invention to the precise form disclosed. Obvious modifications or variations are possible in light of the above teachings. The embodiments were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled. The drawings and preferred embodiments do not and are not intended to limit the ordinary meaning of the claims in their fair and broad interpretation in any way.

What is claimed:

1. A composite material, comprising:
a continuous roving impregnated with a water-based matrix binder.

2. The composite material of claim 1, wherein said continuous roving is made from a material selected from a group consisting of natural fibers, mineral fibers, synthetic fibers, kenaf fibers, hemp fibers, carbon fibers, glass fibers, aramid fibers and mixtures thereof.

3. The composite material of claim 2, wherein said glass fibers are E-glass fibers.

4. The composite material of claim 1, wherein said water-based matrix binder includes an organic material, an inorganic material and water.

5. The composite material of claim 4, wherein said organic material is a polymer and said inorganic material is gypsum.

6. The composite material of claim 5, wherein said polymer is selected from a group consisting of urea formaldehyde resin, melamine formaldehyde resin, phenol-formaldehyde resin, polyvinyl acetate, polyvinyl alcohol, styrene butadiene, acrylic emulsion, urethane and mixtures thereof.

7. The composite material of claim 5, wherein said water based matrix binder includes between about 30 and about 80 weight percent gypsum, between about 0.1 and about 40 weight percent polymer and between about 10 and about 30 weight percent water.

8. The composite material of claim 7, wherein said water based matrix binder also includes between about 0.1 and about 5 weight percent density reducer.

9. The composite material of claim 8, wherein said density reducer is perlite.

10. The composite material of claim 7, wherein said water based matrix binder also includes between about 0.1 and about 0.5 weight percent polyacrylamide.

11. The composite material of claim 7, wherein said water based matrix binder also includes between about 0.1 and about 0.5 weight percent silane coupling agent.

12. The composite material of claim 7, wherein said composite material comprises between about 10 and about 70 weight percent continuous roving and between about 30 and about 90 weight percent water based matrix binder.

13. A method of manufacturing a composite material, comprising:
impregnating a continuous roving made from a material selected from a group consisting of natural fibers, mineral fibers, synthetic fibers, glass fibers, kenaf fibers, hemp fibers, carbon fibers, aramid fibers and mixtures thereof with a water-based matrix binder including an organic material, an inorganic material and water.

14. The method of claim 13, including using a water-based matrix binder including gypsum, polymer and water.

15. The method of claim 13 including using a wet glass fiber roving for said continuous roving and a matrix binder including gypsum, polymer and water for said water-based matrix binder.

16. The method of claim 15, including using said water-based matrix binder with a density between about 1.2 to about 2.0 grams per cubic centimeter.

17. The method of claim 16, including using polyacrylamide in said water-based matrix binder as a processing agent.

18. A method of filament winding a product, comprising:
feeding a continuous roving made from a material selected from a group consisting of natural fibers, mineral fibers, synthetic fibers, glass fibers, kenaf fibers, hemp fibers, carbon fibers, aramid fibers and mixtures thereof impregnated with a water-based matrix binder including an organic material, an inorganic material and water; and
winding said continuous roving over an object selected from a group consisting of a mandrel, a form, a pipe, a vessel, a tank and an epoxy and polymer body.

19. The method of claim 18, including using a wet glass fiber roving for said continuous roving and a matrix binder including gypsum, polymer and water for said water based matrix binder.

20. The method of claim 19, including using said water-based matrix binder with a density between about 1.2 to about 2.0 grams per cubic centimeter.

21. The method of claim 20, including using polyacrylamide in said water-based matrix binder as a processing agent.

22. The method of claim 18, including rotating said object.

23. A method for open molding a product, comprising:
placing a continuous roving made from a material selected from a group consisting of natural fibers, mineral fibers, synthetic fibers, glass fibers, kenaf fibers, hemp fibers, carbon fibers, aramid fibers and mixtures thereof impregnated with a water-based matrix binder including an organic material, an inorganic material and water into an open mold;
allowing said continuous roving to at least partially set in said open mold to form said product; and
removing said product from said open mold.

24. A pultrusion method, comprising:
applying a water-based matrix binder including an organic material, an inorganic material and water to a continuous roving made from a material selected from a group consisting of natural fibers, mineral fibers, synthetic fibers, glass fibers, kenaf fibers, hemp fibers, carbon fibers, aramid fibers and mixtures thereof to form a composite material;
drawing said composite material through a die to form a product having a desired cross-section; and
curing the product.

25. The method of claim 24, including using a wet glass fiber continuous roving as said continuous roving.

26. The method of claim 25, wherein said applying step further includes passing said continuous roving through a tank holding said water-based matrix binder including gypsum, a polymer and water.

27. A method of making a product, comprising:
forming said product from an epoxy filament material; and
over wrapping said product with a continuous roving impregnated with a matrix binder including gypsum, polymer and water.

28. The method of claim 27 wherein said forming includes a filament winding process.

29. The method of claim 28, wherein said overwrapping includes a filament winding process.

30. A pipe comprising:
a tubular body made from an epoxy filament material overwrapped with a continuous roving impregnated with a matrix binder including gypsum, polymer and water.

31. A vessel, comprising:
a hollow body made from an epoxy filament material overwrapped with a continuous roving impregnated with a matrix binder including gypsum, polymer and water.
